(19) United States
(12) Patent Application Publication
(10) Pub. No.: US 2005/0095217 A1
(43) Pub. Date: May 5, 2005

(54) Hair cosmetic compositions

(75) Inventor: Yuji Hirano, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Kao Corporation, Tokyo (JP)

(21) Appl. No.: 10/694,775

(22) Filed: Oct. 29, 2003

(51) Int. Cl. A61K 7/06; A61K 7/075; A61K 7/08

(52) U.S. Cl. 424/70.27; 424/70.31

(57) ABSTRACT

Hair cosmetic compositions contain (A) a higher alcohol, or a fatty acid or its salt; and (B) a diamide compound represented by the following formula (1): 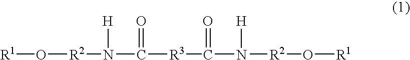
wherein R¹ represents a linear or branched C₁₋₁₂ hydrocarbon group which may be substituted by one or more hydroxyl groups and/or alkoxy groups, R² represents a linear or branched, divalent C₁₋₅ hydrocarbon group, and R³ represents a linear or branched, divalent C₁₋₂₂ hydrocarbon group. The hair cosmetic compositions may protect hair from physical and chemical damages to inhibit occurrence of split ends or broken hair and further, may impart a good feel to the touch such as moisturized feel and silkiness to hair.

### FIELD OF THE INVENTION

This invention relates to hair cosmetic compositions containing amide compounds, which may for example protect hair from physical and chemical damage to inhibit occurrence of split ends or broken hair and further, may impart hair having a good feel to the touch such as a moisturized feel and silkiness.

### BACKGROUND OF THE INVENTION

Hair exists in a damaged state accompanied by a partial loss of its components and structural elements, because it is often exposed to physical damages due to everyday hair care activities, such as dryer heat and brushing friction, and also to chemical damage by permanent wave preparations, hair colors, hair bleaches and/or the like. From the viewpoint of hair care, it is not preferred to leave hair in such a damaged state as it leads to the occurrence of split ends and broken hair, a dry and loose feel, an untidy hairstyle and the like. Protection and/or repair of damaged hair is generally conducted in a way such as supplementing a component or structural element, which has been lost due to the damage, with the corresponding component or structural element or with an analogous substance thereof. For the development of a protecting and/or repairing function, an interaction (compatibility) between a protecting base and hair is considered to be important, and at present, a method making use of a sphingo-lipid or a protein derivative as a protecting base has found wide-spread utility as a beneficial technique. It is, however, the current circumstance that such a base may not be added in any sufficient amount to products from a cost-related consideration despite its recognized effects since the production of such a base requires the need to go through extremely complex steps.

### SUMMARY OF THE INVENTION

In one aspect of the present invention, there is thus provided a hair cosmetic composition which contains diamide compounds. The hair cosmetic composition comprises the folowing ingredients (A) and (B): (A) higher alcohol, or a fatty acid or its salt; and

(B) Diamide Compound Represented by the Following formula (1):

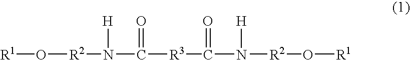

R¹ represents a linear or branched C₁₋₁₂ hydrocarbon group which may be substituted by one or more hydroxyl groups and/or alkoxy groups, R² represents a linear or branched, divalent C₁₋₅ hydrocarbon group, and R³ represents a linear or branched, divalent C₁₋₂₂ hydrocarbon group.

### DETAILED DESCRIPTION OF THE INVENTION

The present invention relates to a hair cosmetic composition containing a base, which may be produced readily and may be supplied at low cost and is effective for the protection and/or repair of hair, and capable of giving an excellent feel in use.

The present inventors have found that certain diamide compounds of a specific structure, which may be produced at low cost and with ease, have excellent effects for preventing split ends and broken hair and further, that hair cosmetic compositions making use of these diamide compounds in combination with higher alcohols or fatty acids may provide a superb feel in use such as moisturized feel and silkiness.

The high alcohol employed as the ingredient (A) in the present invention may be a higher alcohol containing a linear or branched, C₁₂₋₃₀, preferably C₁₂₋₂₄, more preferably C₁₆₋₂₂ alkyl or alkenyl group. Specific examples include cetyl alcohol, stearyl alcohol, isostearyl alcohol, oleyl alcohol, and behenyl alcohol. The aliphatic acid as the ingredient (A) maybe a linear or branched, saturated or unsaturated, C₁₀₋₃₀, preferably C₁₂₋₂₄, more preferably C₁₆₋₂₂ aliphatic acid which may be substituted by one or more hydroxyl groups. Such aliphatic acids also include diacids, triacids and other polyacids. Specific examples may include lauric acid, palmitic acid, stearic acid, behenic acid, oleic acid, elaidic acid, linoleic acid, linolenic acid, arachidonic acid, isostearic acid, coconut fatty acids, beef tallow fatty acids, hydrogenated tallow fatty acids, ricinolic acid, and 12-hydroxystearic acid. The salt of such an aliphatic acid may be an alkali metal or alkaline earth metal salt such as sodium salt, potassium salt or calcium salt, or an amine salt such as triethanolamine salt or monoethanolamine salt.

Two or more of such aliphatic alcohols and fatty acids or fatty acid salts may also be used in combination as the ingredient (A). The content of the ingredient (A) may range preferably from 0.1 to 20 wt. % based on the whole composition, and from the standpoint of obtaining still higher effects, a range of from 0.5 to 15 wt. % is more preferred with a range of from 1 to 10 wt. % being even more preferred, both based on the whole composition.

In formula (1) representing the diamide compound employed as the ingredient (B) in the present invention, R¹ may preferably be a linear or branched C₁₋₁₂ alkyl group which may be substituted by 1 to 3 substituents selected from hydroxyl groups and C₁₋₆ alkoxy groups. Among such alkyl groups, more preferred are unsubstituted C₁₋₁₂ alkyl groups and C₂₋₁₂ alkyl groups each of which is substituted by 1 to 2 hydroxyl groups, one C₁₋₆ alkoxy group, or one hydroxyl group and one C₁₋₆ alkoxy group. Specific examples include methyl, ethyl, propyl, butyl, hexyl, dodecyl, 2-methylpropyl, 2-ethylhexyl, 2-hydroxyethyl, 9-hydroxynonyl, 2,3-dihydroxypropyl, 2-methoxyethyl, 2-hydroxy-3-methoxypropyl, and 9-methoxynonyl. Among these, 2-hydroxyethyl, methyl, dodecyl and 2-methoxyethyl are preferred.

In formula (1), R² may preferably be a linear or branched C₂₋₅, more preferably C₂₋₃ alkylene group. Specific examples include ethylene, trimethylene, tetramethylene, pentamethylene, 1-methylethylene, 2-methylethylene, 1-methyltrimethylene, 2-methyltrimethylene, 1,1-dimethylethylene, and 2-ethyltrimethylene. Among these, ethylene and trimethylene are preferred.

In formula (1), R³ may preferably be a linear or branched, divalent C₂₋₂₂ hydrocarbon group, with a linear or branched C₁₁₋₂₂ alkylene group and a linear or branched C₁₁₋₂₂ alkenylene group having 1 to 4 double bonds being particularly preferred. Specific examples include ethylene, trimethylene, tetramethylene, hexamethylene, heptamethylene, octamethylene, decamethylene, undecamethylene, dodecamethylene, tridecamethylene, tetradecamethylene, hexadecamethylene, octadecamethylene, 1-methylethylene, 2-ethyltrimethylene, 1-methylheptamethylene, 2-methylheptamethylene, 1-butylhexamethylene, 2-methyl-5-ethylheptamethylene, 2,3,6-trimethylheptamethylene, 6-ethyldecamethylene, 7-methyltetradecamethylene, 7-ethylhexadecamethylene, 7,12-dimethyloctadecamethylene, 8,11-dimethyloctadecamethylene, 7,10-dimethyl-7-ethylhexadecamethylene, 1-octadecylethylene, ethenylene, 1-octadecenylethylene, 7,11-octadecadienylene, 7-ethenyl-9-hexadecamethylene, 7,12-dimethyl-7,11-octadecadienylene, and 8,11-dimethyl-7,11-octadecadienylene. Among these, 7,12-dimethyloctadecamethylene, 7,12-dimethyl-7,11-octadecadienylene, octadecamethylene, undecamethylene and tridecamethylene are particularly preferred.

Diamide compounds particularly preferred as the ingredient (B) are those containing the above-exemplified, preferred groups as R¹, R² and R³ in formula (1) in combination. Particularly preferred specific examples of the diamide compound (1) include the following compounds:

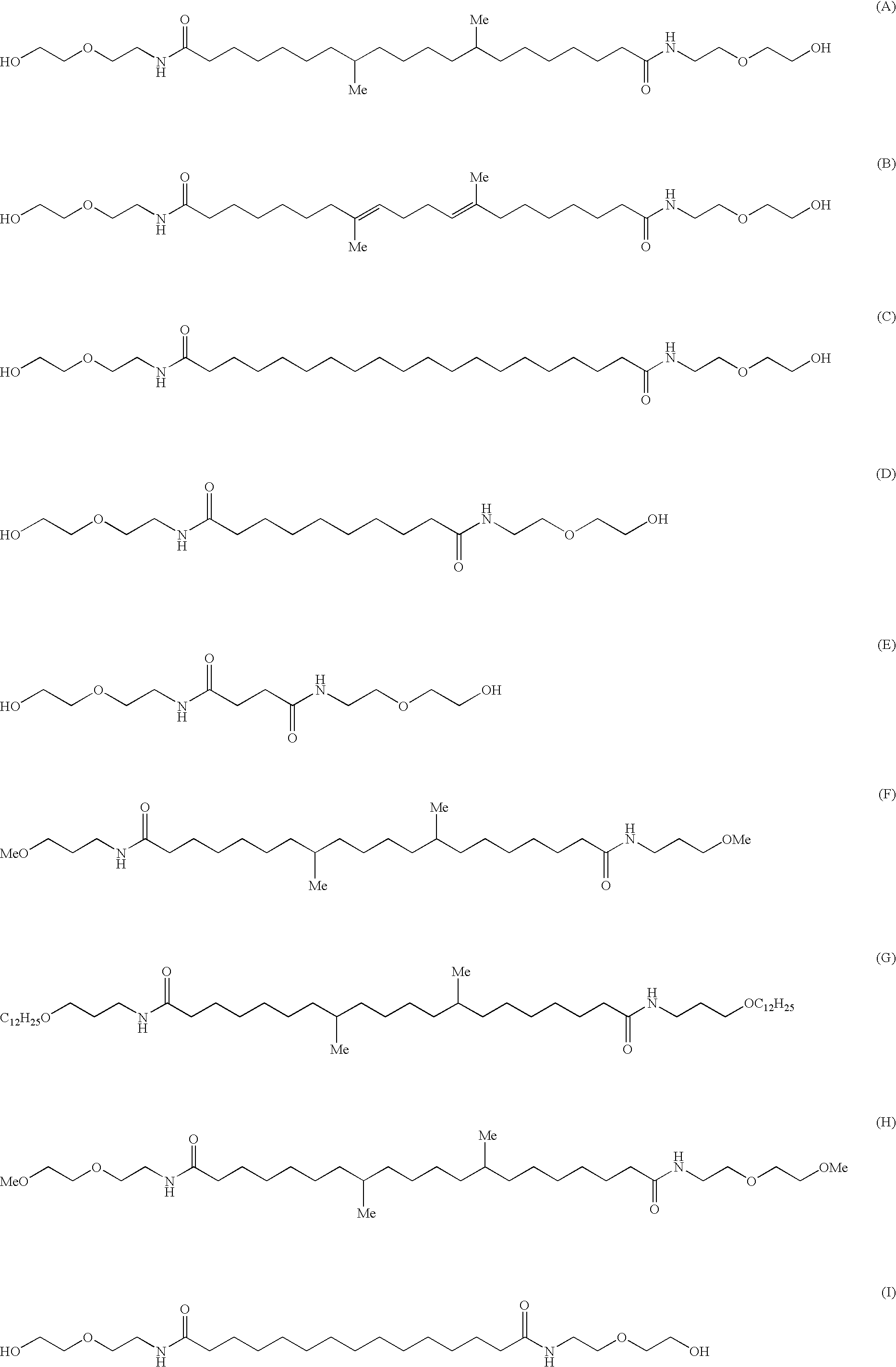

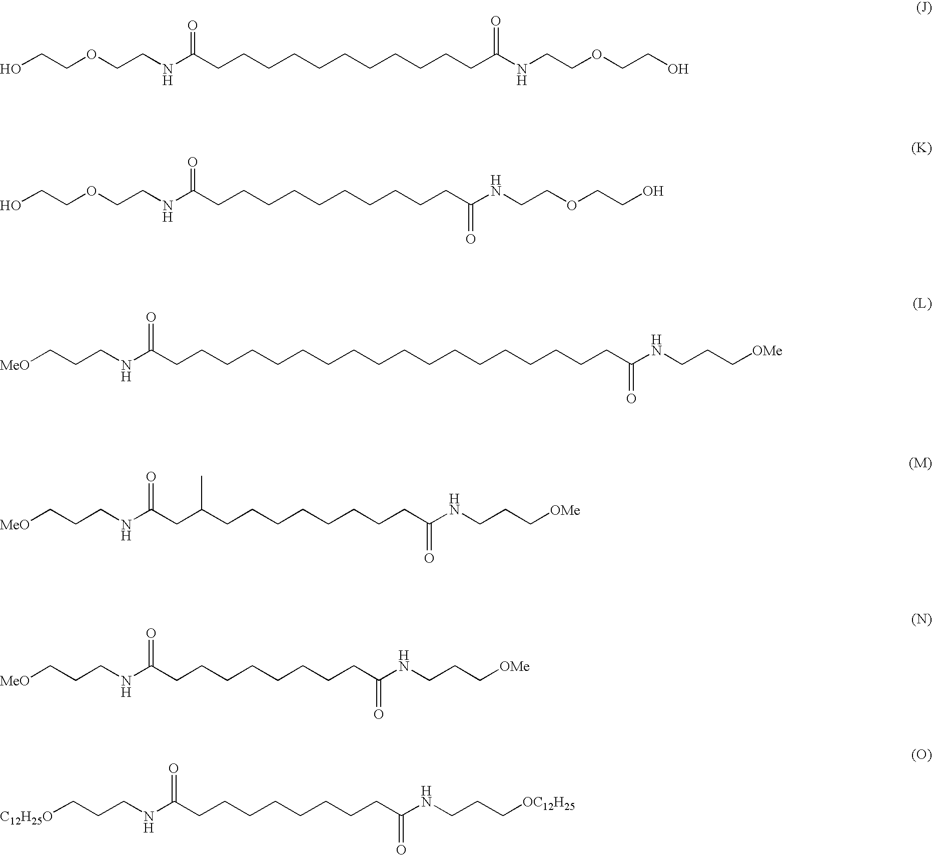

As the ingredient (B), two or more diamide compounds (1) may be used in combination. The content of the ingredient (B) may range preferably from 0.01 to 20 wt. %, more preferably from 0.1 to 20 wt. %, even more preferably from 0.5 to 15 wt. %, all based on the whole composition. Incidentally, these diamide compounds (1) may be synthesized by the process disclosed in the international publication WO 00/61097.

It is preferred to incorporate a surfactant as an ingredient (C) in the hair cosmetic composition according to the present invention with a view to stabilizing the hair cosmetic composition, improving feel in use, controlling its viscosity, and solubilizing, dispersing or emulsifying various bases. As the surfactant, any of a cationic surfactant, an amphoteric surfactant, an anionic surfactant and a nonionic surfactant are usable.

As the cationic surfactant, a quaternary ammonium salt represented by the following formula (2) or an amidoamine represented by the following formula (3) are preferred: 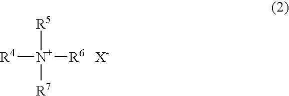
wherein at least one of R⁴, R⁵, R⁶ and R⁷ is a C₈₋₃₀ alkyl or alkenyl group or a C₈₋₂₂ alkoxy, polyoxyalkylene, alkylamido, hydroxyalkyl, aryl or alkylaryl group and the remaining group or groups are each independently a C₁₋₂₂ alkyl, C₂₋₂₂ alkenyl, C₁₋₂₂ alkoxy, C₄₋₂₂ polyoxyalkylene, C₂₋₂₂ alkylamido, C₁₋₂₂ hydroxyalkyl, C₆₋₂₂ aryl or C₇₋₂₂alkylaryl group, and X⁻ represents a halide ion, methosulfate ion or saccharinate ion; 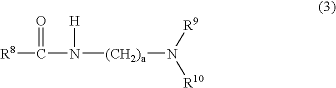
wherein R⁸ represents a linear or branched, C₁₁₋₂₅ alkyl or alkenyl group, a denotes an integer of from 1 to 4, and R⁹ and R¹⁰ each independently represents a hydrogen atom or a C₁₋₄ alkyl or hydroxyalkyl group with a proviso that R⁹ and R¹⁰ are not hydrogen atoms at the same time.

Preferred examples of the quaternary ammonium salt (2) include cetyltrimethylammonium chloride, lauryltrimethylammonium chloride, stearyltrimethylammonium chloride, behenyltrimethylammonium chloride, di(C₁₂₋₁₈ alkyl)dimethylammonium chloride, distearyldimethylammonium chloride, isostearyllauryldimethylammonium chloride, and benzalkonium chloride.

Preferred examples of the amidoamine compound (3) include dimethylaminoethyl palmitamide, diethylaminoethyl palmitamide, dimethylaminopropyl palmitamide, diethylaminopropyl palmitamide, dimethylaminoethyl stearamide, diethylaminoethyl stearamide, dimethylaminopropyl stearamide, diethylaminopropyl stearamide, monoethanolaminoethyl stearamide, diethanolaminoethyl stearamide, dimethylaminoethyl arachidonamide, diethylaminoethyl arachidonamide, dimethylaminopropyl arachidonamide, diethylaminopropyl arachidonamide, dimethylaminoethyl behenamide, diethylaminoethyl behenamide, dimethylaminopropyl behenamide, and diethylaminopropyl behenamide.

Illustrative of the amphoteric surfactant are carbobetaine-type, amidobetaine-type, sulfobetaine-type, hydroxysulfobetaine-type, amidosulfobetaine-type, phosphobetaine-type and imidazolium-type surfactants, each of which contains a C₈₋₂₄ alkyl, alkenyl or acyl group. As counter ions of the anionic groups in these amphoteric surfactants, hydrogen ions, alkali metal ions, alkaline earth metal ions, ammonium ions, alkanolamine ions and the like can be mentioned. As counter ions of the cationic groups in the amphoteric surfactants, on the other hand, halide ions, methosulfate ions, saccharinate ions and the like can be mentioned.

Preferred examples of the amphoteric surfactant may include lauric acid amidopropylbetaine (“ANPHITOL 20AB”, trade name; product of Kao Corporation), coconut fatty acid amidopropylbetaine (“ANPHITOL 55AB”, trade name; product of Kao Corporation), lauryl dimethyl aminoacetic acid betaine (“ANPHITOL 20BS”, trade name; product of Kao Corporation), laurylhydroxysulfobetaine (“ANPHITOL 20H”, trade name; product of Kao Corporation), sodium cocoamphoacetate (“ANPHITOL 20YN”, trade name; product of Kao Corporation) and sodium cocoamphopropionate (“ANPHITOL 20X, Y-B”, trade name; product of Kao Corporation) as 2-alkyl-N-carboxymethyl-N-hydroxyethyl imidazolinium betaines, and acyl-N-carboxyethyl-N-hydroxyethylethylenediamine N-coconut fatty acid sodium salt (“SOFTAZOLINE NS”, trade name; product of Kao Corporation).

Examples of the anionic surfactant include alkyl(or alkenyl) sulfate salts, polyoxyalkylene alkyl (or alkenyl) ether sulfate salts, alkanesulfonate salts, olefinsulfonate salts, alkylbenzenesulfonate salts, alkyl (or alkenyl) sulfosuccinate salts, dialkyl(or dialkenyl)sulfosuccinate salts, polyoxyalkylene alkyl(or alkenyl)sulfosuccinate salts, alkyl(or alkenyl) ether carboxylate salts, polyoxyalkylene alkyl(or alkenyl) ether carboxylate salts, polyoxyalkylene alkyl (or alkenyl) etherphosphate salts, N-acylglutamate salts, N-acyltaurinate salts, and N-acylmethyltaurine. Among these, alkyl(or alkenyl)sulfate salts, polyoxyalkylene alkyl(or alkenyl) ether sulfate salts, alkanesulfonate salts, polyoxyalkylene alkyl(or alkenyl)sulfosuccinate salts, alkyl (or alkenyl) ether carboxylate salts, and polyoxyalkylene alkyl(or alkenyl) ether carboxylate salts are preferred. In these anionic surfactants, each alkyl (or alkenyl) or acyl group may be either linear or branched, and the number of carbon atoms making up such a hydrophobic group may range preferably from 8 to 30, more preferably from 12 to 24, even more preferably from 16 to 22. On the other hand, each oxyalkylene group may preferably be one formed by the addition of ethylene oxide (hereinafter abbreviated as “EO”) or propylene oxide (hereinafter abbreviated as “PO”), and the average number of mole(s) so added may range preferably from 0.2 to 20, more preferably from 0.5 to 15.

Examples of the nonionic surfactant include polyoxyalkylene alkyl (or alkenyl) ethers, each of which has an alkyl or alkenyl group having an average carbon number of from 10 to 20 and contains 1 to 20 moles of EO, PO or butylene oxide (hereinafter abbreviated as “BO”) added thereto; polyoxyalkylene alkyl phenyl ethers, each of which has an alkyl group having an average carbon number of from 6 to 12 and contains 1 to 20 moles of EO or PO added thereto; polyoxyalkylene alkyl (or alkenyl) ethers, each of which has an alkyl or alkenyl group having an average carbon number of from 10 to 20 and contains 1 to 30 moles in total of EO and PO or EO and BO added thereto (EO/PO or EO/BO ratio: 0.1/9.9 to 9.9/0.1); high fatty acid alkanolamides represented by the following formula (4); 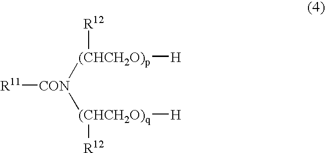
wherein R¹¹ represents a C₇₋₂₁alkyl or alkenyl group, R¹² represents a hydrogen atom or a methyl group, p denotes an integer of from 1 to 3, and q denotes an integer of from 0 to 3, and alkylene oxide adducts thereof; sucrose fatty acid esters formed from fatty acids having an average carbon number of from 10 to 20 and sucrose; and glycerin fatty acid monoesters formed from fatty acids having an average carbon number of from 10 to 20 and glycerin.

Two or more surfactants may be used in combination as the ingredient (C). It is preferred to contain, as the whole part or a part of the ingredient (C), one or more cationic surfactants, more preferably one or more quaternary ammonium salts represented by formula (2) or amidoamine compounds represented by formula (3). The content of the ingredient (C) may range preferably from 0.1 to 20 wt. % and from the standpoint of obtaining still higher effects, from 0.5 to 15 wt. %, more preferably from 1 to 10 wt. %, all based on the whole composition.

For the purpose of making further improvements in the effects for preventing split ends and broken hair, one or more of proteins and ceramides, each of which is commonly employed as a hair protecting ingredient, may be included in the hair cosmetic composition according to the present invention.

Such proteins include protein hydrolysates and derivatives thereof, and can be obtained by extraction from animals or plants or by deriving from the proteins so extracted. Examples of proteins of animal origin include silk protein, keratin, elastine, collagen, lactoferin, casein, α(β)-lactoalbumin, globulins, ovalbumin, silk protein and hydrolysates thereof, with keratin, elastine, collagen, casein, silk protein and hydrolysates thereof being preferred. Examples of proteins of plant origin, on the other hand, include proteins extracted from wheat, malt, oat, barley, corn, rice, soybeans, broadbeans, lupine seeds, potatoes and apricot kernels, and hydrolysates thereof, with wheat protein, soybean protein and hydrolysates being preferred. Two or more proteins may be used in combination. The content of the protein(s) may range preferably from 0.01 to 5 wt. %, more preferably from 0.05 to 4 wt. %, even more preferably from 0.1 to 3 wt. %, all based on the whole composition.

Such ceramides include N-acylated sphingosines, N-acylated phytosphingosines and N-acylated dihydrosphingosines, all of which are available by synthesis or extraction from natural sources. Substituent groups in the acyl substituents on sphingosine, dihydrosphingosine and phytosphingosine may be linear or branched, C₈₋₂₂ alkyl or alkenyl groups, 1 to 5 hydrogen atoms of each of which may be substituted by a like number of hydroxyl groups. For example, in addition to Ceramide 1, Ceramide 2, Ceramide 3, Ceramide 1A, Ceramide 6II and hydroxycaproylphytosphingosine, synthetic pseudo-ceramides such as Sphingo-lipid EX (JP-A-11-209248) and Sphingo-lipid E (JP-B-01-042934) are also usable. Two or more ceramides may be used in combination. The content of the ceramide(s) may range preferably from 0.01 to 5 wt. %, more preferably from 0.05 to 4 wt. %, even more preferably from 0.1 to 3 wt. %, all based on the whole composition.

To further improve the feel in use, the hair cosmetic composition according to the present invention may additionally contain one or more silicone derivatives or cationic polymers which are commonly employed as ingredients for improving feeling to the touch.

Such silicone derivatives include dimethylpolysiloxane, methylphenylpolysiloxane, amino-modified silicones, polyether-modified silicones, epoxy-modified silicones, fluorine-modified silicones, cyclic silicones, alkyl-modified silicones, oxazoline-modified silicones, and the like. Among these, preferred are dimethylpolysiloxane, methylphenylpolysiloxane, amino-modified silicones, polyether-modified silicones, oxazoline-modified silicones, and cyclic silicones. Two or more silicone derivatives may be used in combination. The content of the silicone derivative(s) may range preferably from 0.01 to 20 wt. %, more preferably from 0.05 to 10 wt. %, even more preferably from 0.1 to 5 wt. %, all based on the whole composition.

Such cationic polymers include polydimethyldiallylammonium chloride, acrylamidopropyltrimethylammonium chloride/acrylate copolymers, acrylamide/dimethyldiallylammonium chloride copolymer, methylvinylimidazolinium chloride/vinylpyrrolidone copolymer, hydroxyethylcellulose/diallyldimethylammonium chloride copolymer, diethylsulfate salt of vinylpyrrolidone/dimethylaminoethyl methacrylate copolymer, vinylpyrrolidone/dimethylaminoethyl methacrylate copolymer, vinylpyrrolidone/alkyl aminoacrylate/vinylcaprolactam copolymers, vinylpyrrolidone/dimethylaminopropylmethacrylamide copolymer, O-[2-hydroxy-3-(trimethylammonio)propyl]hydroxycellulose chloride, and guar hydroxypropyltrimonium chloride. Two or more cationic polymers may be used in combination. The content of the cationic polymer(s) may range preferably from 0.01 to 20 wt. %, more preferably from 0.05 to 10 wt. %, evenmore preferably from 0.1 to 5 wt. %, all in terms of solids based on the whole composition.

In addition to the above-described ingredients, the hair cosmetic composition according to the present invention may further contain, depending on its application purpose, one or more oil ingredients such as cholesterols and derivatives thereof, vaseline, lanolin derivatives, and polyethylene glycol fatty acid esters; polymer emulsifiers such as polycarboxylic acids, crosslinked carboxylic acid/carboxylate ester copolymers, crosslinked acrylic acid/acrylate ester copolymers, and acrylamide/acrylamidobutanesulfonic acid copolymer; polyhydric alcohols such as glycerin and sorbitol; humectants; chelating agents such as ethylenediaminetetraacetic acid (EDTA); medicaments such as vitamins; amino acids and derivatives thereof; fine powders of polymers such as polyethylene, polystyrene, poly(methyl methacrylate), nylon and silicones, and hydrophobicization-treated products thereof; animal and plant extracts; ultraviolet absorbers; pearlants; preservatives; antimicrobial agents; anti-inflammatories; antidandruff agents; pH adjusters; dyestuffs; perfumes; and the like.

Illustrative forms of the hair cosmetic composition according to the present invention include those employed in the bathroom, such as hair conditioners, hair treatments and hair packs; and styling hair care products employed outside the bathroom, such as hair milks, hair creams and hair waxes.

### EXAMPLES 1-4 & COMPARATIVE EXAMPLES 1-2

The hair rinses shown in Table 1 were formulated, and then ranked for silkiness and moisturized feel imparted to hair and the percent occurrence of split ends and broken hair. The results are shown in Table 1.

Evaluation Methods

Feel to the Touch (Silkiness and Moisturized Feel)

After each of five expert panelists was shampooed with a commercial shampoo, a rinse (8 g) was applied to the hair. The hair was left standing for 30 seconds and then rinsed with running water. The hair was towel-blotted to remove water, and then dried with hot air from a dryer. With respect to the silkiness and moisturized feel of the thus-dried hair, organoleptic evaluation was performed in accordance with the following standards, and the results were indicated by average scores.

(Silkiness)

5: Silky
4: A little silky
3: Hard to say either way
2: Not very silky
1: Not silky
   (Moisturized Feel to the Touch)
5: Moisturized
4: A little moisturized
3: Hard to say either way
2: Not very moisturized
1: Not moisturized

Percent Occurrence of Split Ends and Broken Hair
1) Flattened hair tresses, each of which was 16 cm in length and was composed of 100 strands of hair, were prepared.
2) The tresses 1) were immersed in individual rinses at room temperature for 10 minutes, any extra portions of the rinses were washed off for 30 minutes in running water, and then, the tresses were dried in air.
3) Brushing stimulation was applied to the thus-rinsed tresses by a motor-driven rotary brush at 100 rpm for about 60 minutes.
4) The tresses were visually observed for tip conditions, and split ends and broken hair occurrence were counted.
5) Assuming that the percent occurrence of split ends and broken hair in a standard tress (Comparative Example 2) was 100%, the percent inhibitions of split ends and broken hair by the respective rinses were determined in accordance with the following formula:
   Percent occurrence of split ends and broken hair=(Number of split ends and broken hair occurred in rinsed hair tress/number of split ends and broken hair occurred in the standard tress)×100

TABLE 1

| | | | | | Comp. | Comp. |
|---|---|---|---|---|---|---|
| (Wt. %) | Ex. 1 | Ex. 2 | Ex. 3 | Ex. 4 | Ex. 1 | Ex. 2 |
| Stearyltri- | 1.0 | 1.0 | 1.0 | 1.0 | 1.0 | 1.0 |
| methyl- | | | | | | |
| ammonium | | | | | | |
| chloride | | | | | | |
| (28 wt. %) | | | | | | |
| Cetanol | 3.0 | 3.0 | 3.0 | 3.0 | — | 3.0 |
| Propylene | 2.0 | 2.0 | 2.0 | 2.0 | 2.0 | 2.0 |
| glycol | | | | | | |
| Diamide | 2.0 | 4.0 | 2.0 | 2.0 | 2.0 | — |
| compound (F) | | | | | | |
| Keratin | — | — | 0.5 | — | — | — |
| hydrolysate¹⁾ | | | | | | |
| Amino- | — | — | — | 0.5 | — | — |
| modified | | | | | | |
| silicone²⁾ | | | | | | |
| Ethyl alcohol | — | — | — | — | 10.0 | — |
| Purified water | Balance | Balance | Balance | Balance | Balance | Balance |
| Silkiness | 3.8 | 4.8 | 4.0 | 4.4 | 2.4 | 1.6 |
| Moisturized | 4.0 | 4.8 | 4.0 | 4.0 | 2.8 | 1.2 |
| feel | | | | | | |
| Percent | 58 | 38 | 51 | 58 | 69 | 100 |
| occurrence of | | | | | | |
| split ends | | | | | | |
| and | | | | | | |
| broken hair | | | | | | |

¹⁾“PROMOIS WK-H”, trade name; product of SEIWA CHEMICAL INDUSTRY CO., LTD.
²⁾“SM8704C”, trade name; product of Dow Corning Toray Silicone Co., Ltd.

### EXAMPLES 5-7 & COMPARATIVE EXAMPLES 1-4

The hair rinses shown in Table 2 were formulated, and in a similar manner as described above, they were evaluated with regard to the percent occurrence of split ends and broken hair. The results are shown in Table 2.

TABLE 2

| | | | | Comp. | Comp. | Comp. | Comp. |
|---|---|---|---|---|---|---|---|
| (Wt. %) | Ex. 5 | Ex. 6 | Ex. 7 | Ex. 1 | Ex. 2 | Ex. 3 | Ex. 4 |
| Stearyltrimethylammonium | 1.0 | 1.0 | 1.0 | 1.0 | 1.0 | 1.0 | 1.0 |
| chloride (28 wt. %) | | | | | | | |
| Cetanol | — | — | — | — | 3.0 | — | — |
| Propylene glycol | 3.0 | 3.0 | 3.0 | 2.0 | 2.0 | 3.0 | 3.0 |
| Diamide | 2.0 | 2.0 | 2.0 | 2.0 | — | — | — |
| compound (F) | | | | | | | |
| Isostearic acid | 2.0 | — | — | — | — | — | — |
| Stearic acid | — | 2.0 | — | — | — | 2.0 | — |
| Behenic acid | — | — | 2.0 | — | — | — | 20 |
| Ethyl alcohol | — | — | — | 10.0 | — | — | — |
| Purified water | Balance | Balance | Balance | Balance | Balance | Balance | Balance |
| Percent occurrence of | 29.4 | 33.5 | 14.7 | 69 | 100 | 70.7 | 71.7 |
| split ends and broken hair | | | | | | | |

### EXAMPLE 8

### Hair Conditioner (pH 3.5)

| | (wt. %) |
|---|---|
| Cetanol | 2.0 |
| Behenyl alcohol | 1.0 |
| Diamide compound (F) | 1.5 |
| Stearyltrimethylammonium chloride¹⁾ | 2.5 |
| Di(C₁₂₋₁₈ alkyl)dimethylammonium chloride²⁾ | 0.5 |
| Polydimethyldiallylammonium chloride³⁾ | 0.2 |
| Polyethylene glycol of high | 0.05 |
| polymerization degree⁴⁾ | |
| Isopropyl palmitate | 0.5 |
| Diethylene glycol monoethyl ether | 0.5 |
| Benzyloxy ethanol | 0.5 |
| Hydroxyethylcellulose | 0.2 |
| Aq. soln. of malic acid (50 wt. %) | q.s. |
| Perfume | q.s. |
| Purified water | Balance |
| Total | 100.0 |

¹⁾“QUARTAMIN 86W”, trade name; product of Kao Corporation (28 wt. %)
²⁾“QUARTAMIN D2345P”, trade name; product of Kao Corporation (75 wt. %)
³⁾“MERQUAT 100”, trade name; product of Calgon Corp.
⁴⁾“POLYOX WSRN-60K”, trade name; product of UNION CARBIDE JAPAN K.K.

### EXAMPLE 9

### Hair Treatment (pH 3.5)

| | (wt. %) |
|---|---|
| Behenic acid | 5.0 |
| Stearic acid | 3.0 |
| Diamide compound (A) | 2.0 |
| Diamide compound (D) | 1.0 |
| Cetyltrimethylammonium chloride¹⁾ | 8.5 |
| Di(C₁₂₋₁₈ alkyl)dimethylammonium chloride²⁾ | 0.5 |
| Keratin hydrolysate³⁾ | 0.2 |
| Ceramide 3⁴⁾ | 0.1 |
| Amino-modified silicone emulsion⁵⁾ | 0.2 |
| Dipentaerythritol fatty acid ester⁶⁾ | 0.3 |
| Hydroxymethoxybenzophenonesulfonic acid | 0.3 |
| Diethylene glycol monoethyl ether | 1.0 |
| Benzyloxyethanol | 0.5 |
| Hydroxyethylcellulose | 0.4 |
| Aq. soln. of citric acid (50 wt. %) | q.s. |
| Perfume | q.s. |
| Purified water | Balance |
| Total | 100.0 |

¹⁾“QUARTAMIN 60W”, trade name; product of Kao Corporation (30 wt. %)
²⁾“QUARTAMIN D2345P”, trade name; product of Kao Corporation (75 wt. %)
³⁾“PROMOIS WK-H”, trade name; product of SEIWA CHEMICAL INDUSTRY CO., LTD.
⁴⁾“Ceramide III”, trade name; product of Gist-brocades/Cosmoferm BV
⁵⁾“SM8704C”, trade name; product of Dow Corning Toray Silicone Co., Ltd.
⁶⁾“COSMOL 168AR”, trade name; product of The Nisshin OilliO, Ltd.

### EXAMPLE 10

### Hair Conditioner (pH 5.5)

| | (wt. %) |
|---|---|
| Cetanol | 3.0 |
| Behenyl alcohol | 1.5 |
| Oleyl alcohol | 0.5 |
| Diamide compound (N) | 4.0 |
| Stearamidoethyl diethylamine | 1.8 |
| Amino-modified silicone emulsion¹⁾ | 0.2 |
| Polyethylene glycol of high | 0.05 |
| polymerization degree²⁾ | |
| Dipentaerythritol fatty acid ester³⁾ | 1.0 |
| Liquid paraffin | 0.5 |
| Hydroxyethylcellulose | 0.1 |
| Lactic acid | q.s. |
| Perfume | q.s. |
| Purified water | Balance |
| Total | 100.0 |

¹⁾“SM8704C”, trade name; product of Dow Corning Toray Silicone Co., Ltd.
²⁾“POLYOX WSRN-60K”, trade name; product of UNION CARBIDE JAPAN K.K.
³⁾“COSMOL 168AR”, trade name; product of The Nisshin OilliO, Ltd.

### EXAMPLE 11

### Hair Treatment (pH 4.5)

| | (wt. %) |
|---|---|
| Behenic acid | 4.5 |
| Stearic acid | 3.0 |
| Diamide compound (F) | 3.0 |
| Stearamidoethyl diethylamine | 2.5 |
| Di(C₁₂₋₁₈ alkyl)dimethylammonium chloride¹⁾ | 0.5 |
| Collagen hydrolysate²⁾ | 0.3 |
| Ceramide 1³⁾ | 0.1 |
| Polydimethyldiallylammonium chloride⁴⁾ | 0.2 |
| Dipentaerythritol fatty acid ester⁵⁾ | 0.3 |
| 2-Ethylhexyl paramethoxycinnamate | 0.15 |
| Diethylene glycol monoethyl ether | 1.0 |
| Benzyloxyethanol | 0.5 |
| Hydroxyethylcellulose | 0.4 |
| Aq. soln. of citric acid | q.s. |
| Perfume | q.s. |
| Purified water | Balance |
| Total | 100.0 |

¹⁾“QUARTAMIN D2345P”, trade name; product of Kao Corporation (75 wt. %)
²⁾“PROMOISE-118D”, trade name; product of SEIWA CHEMICAL INDUSTRY CO., LTD.
³⁾“Phytoceramide 1”, trade name; product of Gist-brocades/Cosmoferm BV
⁴⁾“MERQUAT 100”, trade name; product of Calgon Corp.
⁵⁾“COSMOL 168AR”, trade name; product of The Nisshin OilliO, Ltd.

With respect to the individual compositions of Examples 8-11, organoleptic evaluation was performed by expert panelists in a similar manner as in Example 1. As a result, it was confirmed that all the compositions imparted a good feel to hair upon drying and, moreover, that the feeling lasted.

### EXAMPLE 12

### Hair Styling Cream

| | (wt. %) |
|---|---|
| Cetanol | 5.0 |
| Octyl dodecanol | 1.0 |
| Diamide compound (F) | 2.0 |
| Hydrolyzed silk liquid¹⁾ | 0.5 |
| Polyoxyethylene-methylpolysiloxane | 2.0 |
| copolymer²⁾ | |
| Sodium POE stearyl ether phosphate³⁾ | 0.5 |
| Glyceryl monoisostearate monomyristate | 7.0 |
| Glycerin | 5.0 |
| Carboxyvinyl polymer⁴⁾ | 0.15 |
| Disodium edetate | 0.1 |
| Diethylene glycol monoethyl ether | 1.0 |
| Butyl paraoxybenzoate | 0.3 |
| Methyl paraoxybenzoate | 0.2 |
| 48 wt. % sodium hydroxide | 0.15 |
| Perfume | q.s. |
| Purified water | Balance |
| Total | 100.0 |

¹⁾“Silkgen G Soluble KE”, trade name; product of ICHIMARU PHARCOS Co., Ltd.
²⁾“SILICONE SH-3775M”, trade name; product of Dow Corning Toray Silicone Co., Ltd.
³⁾“SPE-104NB”, trade name; product of Kao Corporation
⁴⁾“Carbopol 941”, trade name; product of BF Goodrich Co.

### EXAMPLE 13

### Hair Styling Wax

| | (wt. %) |
|---|---|
| Cetanol | 7.0 |
| Stearic acid | 1.2 |
| Palmitic acid | 0.8 |
| Diamide compound (N) | 4.0 |
| Sodium POE stearyl ether phosphate¹⁾ | 0.1 |
| N-Stearoyl-N-methyltaurine sodium salt | 0.4 |
| Polyoxyethylene(20) sorbitan monostearate | 2.0 |
| Sorbitan monostearate | 3.5 |
| Sphingo-lipid EX²⁾ | 0.1 |
| Methylpolysiloxane (5 cs) | 5.0 |
| Liquid paraffin | 8.0 |
| Diisostearyl malate | 8.0 |
| Isostearyl glyceryl ether | 1.0 |
| Polyethylene glycol of high | 0.15 |
| polymerization degree³⁾ | |
| Sorbitol solution (70 wt. %) | 10.0 |
| Glycerin | 15.0 |
| Carboxyvinyl polymer⁴⁾ | 0.1 |
| Disodium edetate | 0.05 |
| 48 wt. % sodium hydroxide | q.s. |
| Butyl paraoxybenzoate | 0.1 |
| Methyl paraoxybenzoate | 0.3 |
| Perfume | 0.1 |
| Ethyl alcohol | 3.0 |
| Purified water | Balance |
| Total | 100.0 |

¹⁾“SPE-104NB”, trade name; product of Kao Corporation
²⁾“AQUACERAMIDE”, trade name; product of Kao Corporation
³⁾“POLYOX WSRN-60K”, trade name; product of UNION CARBIDE JAPAN K.K.
⁴⁾“Carbopol 981”, trade name; product of BF Goodrich Co.

1. A hair cosmetic composition comprising the following ingredients (A) and (B):
(A) a higher alcohol, or a fatty acid or a salt thereof; and
(B) a diamide compound represented by the following formula (1): 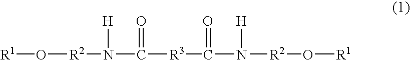
Wherein R¹ represents a linear or branched C₁₋₁₂ hydrocarbon group which may be substituted by one or more hydroxyl groups and/or alkoxy groups, R² represents a linear or branched, divalent C₁₋₅ hydrocarbon group, and R³ represents a linear or branched, divalent C₁₋₂₂ hydrocarbon group.

2. A hair cosmetic composition according to claim 1, comprising as ingredient (B) a diamide compound of formula (1) in which R³ is a linear or branched C₁₁₋₂₂ alkylene group or a linear or branched C₁₁₋₂₂ alkenylene group having 1 to 4 double bonds.

3. A hair cosmetic composition according to claim 1 or 2, further comprising a surfactant as ingredient (C).

4. A hair cosmetic composition according to claim 3, comprising one or more cationic surfactants as the whole or a part of ingredient (C).
